(19) United States
(12) Patent Application Publication
(10) Pub. No.: US 2026/0021558 A1
(43) Pub. Date: Jan. 22, 2026

(54) METHOD OF MANUFACTURING MEDICAL DEVICE

(71) Applicant: OLYMPUS MEDICAL SYSTEMS CORP., Tokyo (JP)

(72) Inventor: Tsunetaka Akagane, Tokyo (JP)

(21) Appl. No.: 19/035,078

(22) Filed: Jan. 23, 2025

Related U.S. Application Data
(63) Continuation of application No. PCT/JP2024/025702, filed on Jul. 17, 2024.

(51) Int. Cl. B24C 1/10; A61B 17/00; A61B 17/32; B29C 71/00; B29L 31/00

(52) U.S. Cl. CPC B24C 1/10; A61B 17/320092; B29C 71/0072; A61B 2017/00526; A61B 2017/320074; A61B 2017/320082; A61B 2017/320095; B29L 2031/753

(57) ABSTRACT

Provided is a method of manufacturing a medical device that is repeatedly used by performing cleaning, disinfection, or sterilization using a medical agent. The method includes: performing first blasting on an outer surface that is included in the medical device and is made of a resin material to change an orientation of a fibril of the resin material.

### CROSS REFERENCES TO RELATED APPLICATIONS

This application is a continuation of International Application No. PCT/JP2024/025702, filed on Jul. 17, 2024, the entire contents of which are incorporated herein by reference.

### BACKGROUND

### 1. Technical Field

The present disclosure relates to a method of manufacturing a medical device.

### 2. Related Art

In the related art, there is a medical device that is repeatedly used by performing cleaning, disinfection, or sterilization using a medical agent (see, for example, JP 2013-81776 A and JP 2019-521793 W.).

The medical devices described in JP 2013-81776 A and JP 2019-521793 W are transducers of ultrasonic treatment devices. The ultrasonic treatment device treats a treatment target by supplying ultrasonic vibration generated by the transducer according to the supplied power to a treatment target site (hereinafter, described as a treatment target) in a biological tissue.

FIGS. 6A and 6B are diagrams illustrating a conventional problem.

Incidentally, a resin portion made of a resin material is often used for a medical device repeatedly used.

As illustrated in FIG. 6A, there may be a craze 110 generated by the action of stress on the surface of a resin portion 100. As illustrated in FIG. 6B, the craze 110 includes fibrils 120 oriented in a stress direction (a direction indicated by an arrow in FIG. 6A and a vertical direction in FIG. 6B) and minute voids 130. In FIG. 6B, an orientation of the fibrils 120 is represented by a straight line. As described above, when the orientation of the fibrils 120 is uniform, when cleaning, disinfection, or sterilization is performed using a medical agent, the medical agent acts, and the fibrils 120 may be broken and grow into solvent cracks. Even when the craze 110 is not present, when the orientation of the fibrils 120 is uniform, cleaning, disinfection, or sterilization is performed using a medical agent, the medical agent acts, and the fibrils 120 may be broken and grow into solvent cracks.

Therefore, there is a demand for a technique capable of suppressing the occurrence of solvent cracks.

### SUMMARY

In some embodiments, provided is a method of manufacturing a medical device that is repeatedly used by performing cleaning, disinfection, or sterilization using a medical agent. The method includes: performing first blasting on an outer surface that is included in the medical device and is made of a resin material to change an orientation of a fibril of the resin material.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an example of a medical device according to an embodiment;

FIG. 2 is a flowchart illustrating a method of manufacturing the medical device according to the embodiment;

FIGS. 3A and 3B are diagrams for describing a method of manufacturing a medical device;

FIG. 4 is a diagram for describing evaluation results of samples of the first comparative example and the first example to the fifth example;

FIG. 5 is a diagram illustrating a modification of the embodiment; and

FIGS. 6A and 6B are diagrams for describing a conventional problem.

### DETAILED DESCRIPTION

Hereinafter, embodiments will be described with reference to the drawings. Note that the disclosure is not limited by the embodiments described below. Further, in the description of the drawings, the same parts will be described with the same reference numerals.

### Schematic Configuration of Medical Device

Before describing the method of manufacturing the medical device according to the present embodiment, a medical device that is a target of the manufacturing method will be described.

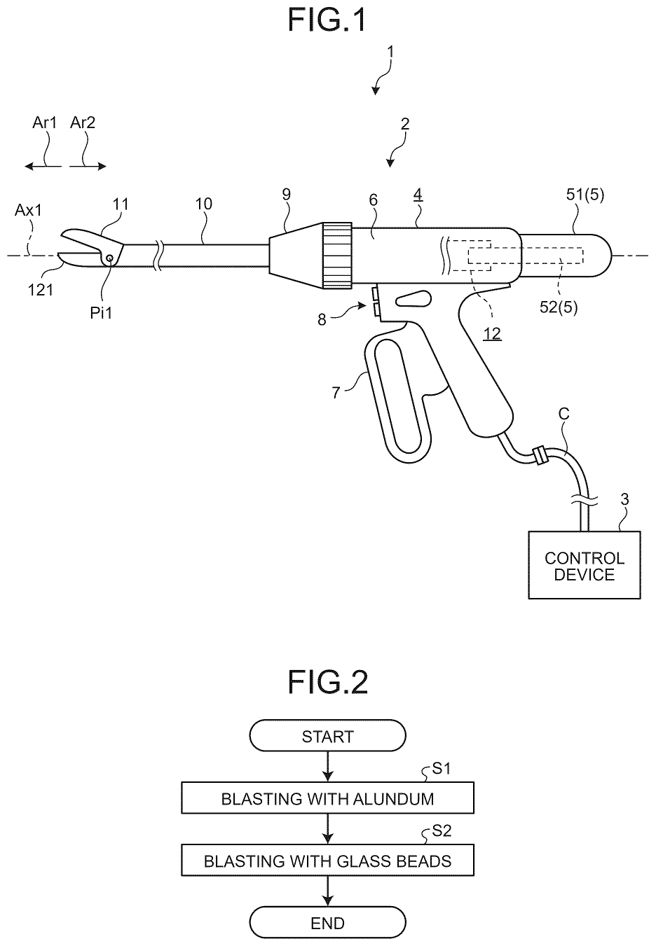

FIG. 1 is a diagram illustrating an example of a medical device according to an embodiment. Specifically, FIG. 1 is a diagram illustrating a treatment system 1.

The treatment system 1 treats a treatment target by applying treatment energy to a treatment target site (hereinafter, described as a treatment target) in a living tissue. The treatment energy in the present embodiment is ultrasonic energy and high frequency energy. In addition, the treatment that can be performed by the treatment system 1 according to the present embodiment is treatment such as coagulation (sealing) of a treatment target or incision of a treatment target. Coagulation and incision may be performed simultaneously. As illustrated in FIG. 1, the treatment system 1 includes a treatment device 2 and a control device 3.

### Configuration of Treatment Device

Hereinafter, one side of an outer pipe 10 along the center axis Ax1 (FIG. 1) is described as a distal end side Ar1, and the other side is described as a proximal end side Ar2.

The treatment device 2 is an ultrasonic treatment device. Then, the treatment device 2 treats the treatment target by applying ultrasonic energy and high frequency energy to the treatment target. As illustrated in FIG. 1, the treatment device 2 includes a handpiece 4 and a transducer 5.

As illustrated in FIG. 1, the handpiece 4 includes a fixing handle 6, an operation handle 7, a switch 8, a rotary knob 9, an outer pipe 10, a jaw 11, and an ultrasonic blade 12.

The fixing handle 6 is a portion that supports the entire treatment device 2 and is gripped by an operation person (user) such as an operator.

The operation handle 7 is movably attached to the fixing handle 6 and receives an opening/closing operation by an operation person such as an operator.

The switch 8 is provided in a state of being exposed to the outside of the fixing handle 6, and receives a treatment operation by an operation person such as an operator.

The rotary knob 9 has a substantially cylindrical shape coaxial with the center axis Ax1, and is provided on the distal end side Ar1 of the fixing handle 6. Then, the rotary knob 9 receives a rotation operation by an operation person such as an operator. By the rotation operation, the rotary knob 9 rotates about the center axis Ax1 with respect to the fixing handle 6. The outer pipe 10, the jaw 11, and the ultrasonic blade 12 rotate about the center axis Ax1 by the rotation of the rotary knob 9.

The outer pipe 10 has a tubular shape. In the present embodiment, the outer pipe 10 is a cylindrical pipe made of an electrically conductive material such as metal.

In the outer pipe 10, a first pin Pi1 having a columnar shape extending in a direction orthogonal to the paper surface of FIG. 1 and engaging with the jaw 11 and pivotally supporting the jaw 11 in a rotatable manner is fixed to an end portion on the distal end side Ar1.

The outer peripheral face of the outer pipe 10 is covered with an electrically insulating outer tube (not illustrated). Further, a tubular inner pipe (not illustrated) that moves forward and backward along the longitudinal direction of the outer pipe 10 in response to an opening/closing operation on the operation handle 7 by an operation person such as an operator is inserted into the outer pipe 10. A second pin (not illustrated) having a columnar shape extending in a direction orthogonal to the paper surface of FIG. 1 and engaging with the jaw 11 is fixed to an end portion of the inner pipe on the distal end side Ar1.

The jaw 11 is connected to the outer pipe 10 via the first pin Pi1. The jaw 11 is connected to the inner pipe by the second pin. The jaw 11 rotates about the first pin Pi1 with respect to the outer pipe 10 in conjunction with forward and backward movement of the inner pipe according to the opening/closing operation on the operation handle 7 by the operation person such as an operator. As a result, the jaw 11 is opened from and closed to a treatment unit 121 which is the end portion of the ultrasonic blade 12 on the distal end side, and can grip the treatment target between the jaw and the treatment unit 121.

Note that the treatment device 2 may be a push-close type or a pull-close type.

The push-close type has the following configuration.

The jaw 11 rotates about the first pin Pi1 in a direction approaching the treatment unit 121 in conjunction with the movement of the inner pipe toward the distal end side Ar1 described above. That is, the jaw 11 is closed to the treatment unit 121. The jaw 11 rotates about the first pin Pi1 in a direction away from the treatment unit 121 in conjunction with the movement of the inner pipe toward the proximal end side Ar2. That is, the jaw 11 is opened to the treatment unit 121.

The pull-close type has the following configuration.

The jaw 11 rotates about the first pin Pi1 in a direction approaching the treatment unit 121 in conjunction with the movement of the inner pipe toward the proximal end side Ar2 described above. That is, the jaw 11 is closed to the treatment unit 121. The jaw 11 rotates about the first pin Pi1 in a direction away from the treatment unit 121 in conjunction with the movement of the inner pipe toward the distal end side Ar1. That is, the jaw 11 is opened to the treatment unit 121.

A detailed configuration of the jaw 11 will be described in “Configuration of jaw” described later.

The ultrasonic blade 12 is made of an electrically conductive material and has an elongated shape extending along the center axis Ax1. In addition, the ultrasonic blade 12 is inserted into the inner pipe described above in a state where the treatment unit 121 protrudes to the outside. At this time, an end portion of the ultrasonic blade 12 on the proximal end side Ar2 is mechanically connected to an ultrasonic transducer 52 constituting the transducer 5 as illustrated in FIG. 1. Then, the ultrasonic blade 12 transmits the ultrasonic vibration generated by the transducer 5 from the end portion on the proximal end side Ar2 to the treatment unit 121. The ultrasonic vibration is longitudinal vibration that vibrates in a direction along the center axis Ax1. In the ultrasonic blade 12, the outer peripheral face excluding the treatment unit 121 is covered with an electrically insulating inner tube.

The transducer 5 corresponds to a medical device. That is, the transducer 5 is a medical device repeatedly used by performing cleaning, disinfection, or sterilization using a medical agent. Note that the medical device is not limited to the transducer 5, and other medical devices may be used. As illustrated in FIG. 1, the transducer 5 includes a TD (transducer) case 51 and the ultrasonic transducer 52.

The TD case 51 supports the ultrasonic transducer 52 and is detachably connected to the fixing handle 6. In the present embodiment, the TD case 51 is made of a resin material. Examples of the resin material include polyphenylsulfone or polyetheretherketone.

The ultrasonic transducer 52 generates ultrasonic vibration according to the supplied power under the control of the control device 3. In the present embodiment, the ultrasonic transducer 52 is configured by a bolted Langevin transducer (BLT).

### Configuration of Control Device

The control device 3 integrally controls the operation of the treatment device 2 via the electric cable C (FIG. 1).

Specifically, the control device 3 detects a treatment operation on the switch 8 by an operation person such as an operator via the electric cable C. Then, when detecting the treatment operation, the control device 3 applies treatment energy to the treatment target gripped between the jaw 11 and the treatment unit 121 via the electric cable C. That is, the control device 3 treats the treatment target.

For example, when applying ultrasonic energy to a treatment target, the control device 3 supplies drive power to the ultrasonic transducer 52 via the electric cable C. As a result, the ultrasonic transducer 52 generates longitudinal vibration (ultrasonic vibration) that vibrates in a direction along the center axis Ax1. In addition, the treatment unit 121 vibrates with a desired amplitude in the longitudinal vibration. Then, ultrasonic vibration is supplied from the treatment unit 121 to the treatment target gripped between the jaw 11 and the treatment unit 121. Ultrasonic energy is applied from the treatment unit 121 to the treatment target.

In addition, for example, when applying high frequency energy to the treatment target, the control device 3 supplies high frequency power between the jaw 11 and the ultrasonic blade 12 via the electric cable C or the like. When the high frequency power is supplied between the jaw 11 and the ultrasonic blade 12, a high frequency current is supplied to the treatment target gripped between the jaw 11 and the treatment unit 121. In other words, high frequency energy is applied to the treatment target.

### Method of Manufacturing Medical Device

Next, a method of manufacturing a medical device such as the transducer 5 described above will be described. Note that, in the following, only steps for main parts of the method of manufacturing the medical device will be described as a method of manufacturing a medical device such as the transducer 5.

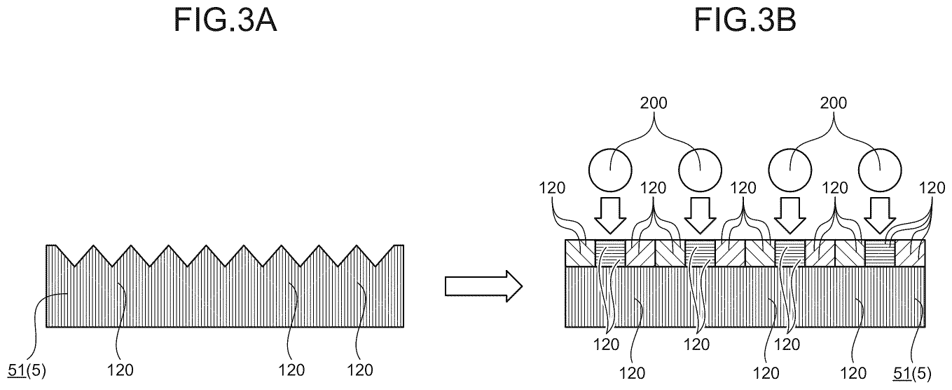

FIG. 2 is a flowchart illustrating a method of manufacturing the medical device according to the embodiment. FIGS. 3A and 3B are diagrams illustrating a method of manufacturing a medical device. Specifically, FIGS. 3A and 3B illustrate an outer surface of the TD case 51. In FIGS. 3A and 3B, an orientation of the fibrils 120 is represented by a straight line.

First, the worker performs the following step (Step S1).

Step S1 is a step of performing blasting using alundum on the outer surface of the TD case 51 made of a resin material. The blasting corresponds to the second blasting. Furthermore, the alundum is a medium with a corner and corresponds to the second medium. Then, by performing Step S1, as illustrated in FIG. 3A, the outer surface of the TD case 51 is scraped by collision of the alundum and has an uneven shape.

Here, the granularity of alundum used in the blasting in Step S1 is preferably #1000 or more.

After Step S1, the worker performs the following step (Step S2).

Step S2 is a step of performing blasting using glass beads 200 on the outer surface of the TD case 51 made of a resin material. The blasting corresponds to the first blasting. Then, by performing Step S2, as illustrated in FIG. 3B, the glass beads 200 collide with the outer surface of the TD case 51, so that the orientation of the fibrils 120 originally in a longitudinal direction changes to a lateral direction, and voids (for example, the voids 130 in FIG. 6B) are crushed.

Here, the granularity of the glass beads 200 used in the blasting in Step S2 is preferably #100 or more. A particle diameter of each glass bead 200 is preferably larger than a particle diameter of the alundum used in the blasting in Step S1.

The medium used in the blasting in Step S2 is not limited to the glass bead 200, and other medium may be used as long as the medium has no corner.

According to the present embodiment described above, the following effects are obtained.

The method of manufacturing the medical device according to the present embodiment includes a step (Step S2) of performing first blasting on an outer surface that is included in the medical device and is made of a resin material to change the orientation of the fibrils 120.

Therefore, according to the method of manufacturing the medical device according to the present embodiment, it is possible to suppress an occurrence of solvent cracks caused by the alignment of the orientation of the fibrils 120 in the medical device.

Specifically, in the first blasting, the glass bead 200 which is a medium without corner is used. Therefore, it is possible to efficiently change the orientation of the fibrils 120 and crush the voids 130 without scraping the outer surface of the medical device.

### EXAMPLES

Next, effects of the disclosure will be described based on specific examples.

### First Comparative Example

The sample of the first comparative example is a sample in which no processing is performed on the outer surface of the TD case 51 made of a resin material.

### First Example

The sample of the first example is a sample obtained by performing only a treatment (hereinafter, described as a heat treatment) of leaving the sample at 200° C. for 8 hours with the outer surface of the TD case 51 made of a resin material exposed.

### Second Example

The sample of the second example is a sample obtained by performing only the blasting in Step S2 described above on the outer surface of the TD case 51 made of a resin material.

### Third Example

The sample of the third example is a sample obtained by performing the above-described heat treatment on the outer surface of the TD case 51 made of a resin material and then performing blasting in Step S2 described above.

### Fourth Example

The sample of the fourth example is a sample obtained by performing blasting in Step S1 described above on the outer surface of the TD case 51 made of a resin material and then performing blasting in Step S2 described above.

### Fifth Example

The sample of the fifth example is a sample obtained by performing the above-described heat treatment on the outer surface of the TD case 51 made of a resin material, then performing blasting in Step S1 described above, and further performing blasting in Step S2 described above.

### Evaluation Results

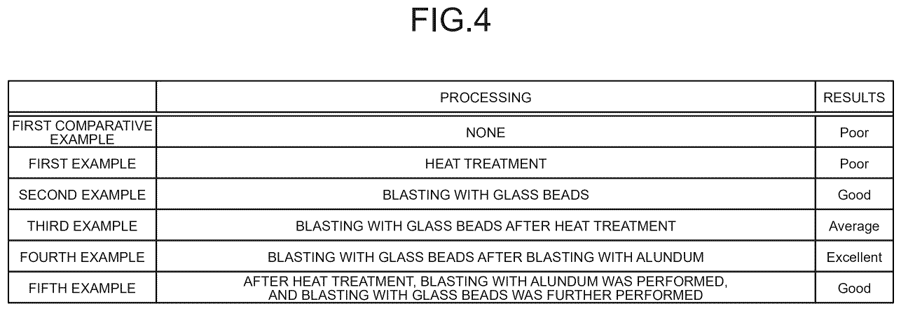

FIG. 4 is a diagram illustrating evaluation results of samples of the first comparative example and the first example to the fifth example.

The samples of the first comparative example and the first example to the fifth example were immersed in a medical agent such as a disinfectant solution, the samples were taken out each time a predetermined time elapsed, and whether a solvent crack occurred was evaluated with a microscope. In FIG. 4, the sample in which the solvent crack occurred in a short immersion time was evaluated as “Poor”, and as the immersion time required for the occurrence of the solvent crack was longer than that of the evaluation with “Poor”, the evaluation was made in the order of “Average” and “Good”. A sample in which a solvent crack did not occur even when the immersion time was long was evaluated as “Excellent”.

As a result, as can be seen from the evaluation results of the samples of the second, fourth and fifth example, it was found that the occurrence of solvent cracks can be suppressed by performing blasting using the glass beads 200. In addition, as can be seen from the evaluation results of the sample of the fourth example, it was found that performing blasting using the glass beads 200 after performing blasting using alundum is most effective for suppressing the occurrence of solvent cracks.

### Other Embodiments

The present disclosure should not be limited simply by the above-described embodiments.

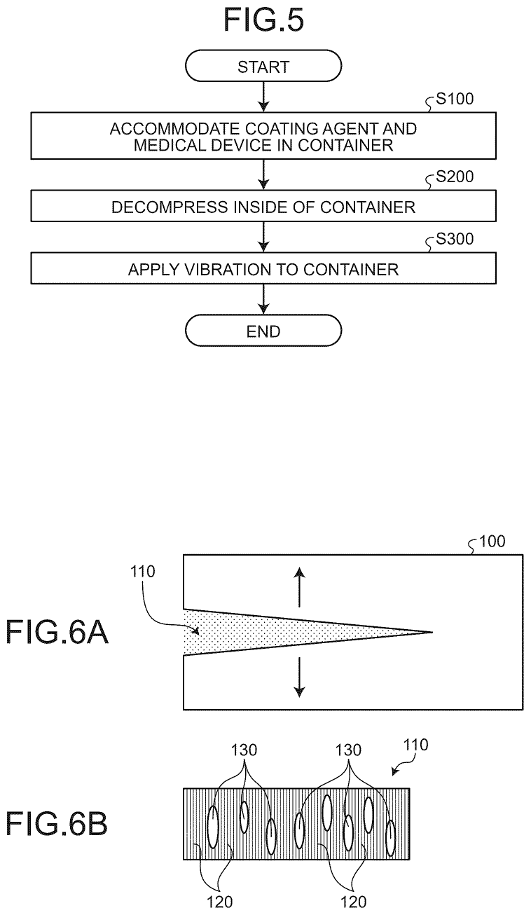

FIG. 5 is a view for describing a modification of the embodiment. Specifically, FIG. 5 is a flowchart illustrating a modification of the method of manufacturing the medical device.

In order to suppress the occurrence of solvent cracks, the method of manufacturing a medical device illustrated in FIG. 5 may be used.

First, the worker performs the following step (Step S100).

Step S100 is a step of accommodating a coating agent and a medical device such as the transducer 5 whose outer surface is made of a resin material in a predetermined container. An examples of the coating agent include a fluorine-based monomolecular water repellent agent.

After Step S100, the worker performs the following step (Step S200).

Step S200 is a step of decompressing an inside of the container described above.

After Step S200, the worker performs the following step (Step S300).

Step S300 is a step of applying vibration to the container described above.

By performing Step S200, the volume of air in the void (for example, the void 130 in FIG. 6B) included in the medical device stored in the container described above increases. Then, when the air comes out from the outer surface of the medical device, Step S300 is performed to allow the coating agent to permeate the inside of the medical device. This makes it possible to suppress the occurrence of solvent cracks.

According to the method of manufacturing the medical device according to the disclosure, it is possible to suppress the occurrence of solvent cracks.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method of manufacturing a medical device that is repeatedly used by performing cleaning, disinfection, or sterilization using a medical agent, the method comprising:
performing first blasting on an outer surface that is included in the medical device and is made of a resin material to change an orientation of a fibril of the resin material.

2. The method of manufacturing the medical device according to claim 1, wherein
the first blasting is performed using a medium without a corner.

3. The method of manufacturing the medical device according to claim 2, wherein
the medium is a glass bead.

4. The method of manufacturing the medical device according to claim 3, wherein
granularity of the glass bead is #100 or more.

5. The method of manufacturing the medical device according to claim 1, wherein
the first blasting is performed to crush a void in the outer surface in addition to the changing the orientation of the fibril.

6. The method of manufacturing the medical device according to claim 1, further comprising:
performing, before the first blasting, second blasting on the outer surface of the medical device using a second medium different from a first medium used for the first blasting.

7. The method of manufacturing the medical device according to claim 6, wherein
the second blasting is performed using the second medium with a corner.

8. The method of manufacturing the medical device according to claim 6, wherein
the second medium is alundum.

9. The method of manufacturing the medical device according to claim 6, wherein
a particle diameter of the second medium is smaller than a particle diameter of the first medium used for the first blasting.

10. The method of manufacturing the medical device according to claim 6, wherein
granularity of the second medium is #1000 or more.

11. The method of manufacturing the medical device according to claim 1, wherein
the resin material is polyphenylsulfone or polyetheretherketone.

12. The method of manufacturing the medical device according to claim 1, wherein
the medical device is a transducer used in an ultrasonic treatment device and configured to generate ultrasonic vibration according to supplied power.
